(19) United States
(12) Patent Application Publication
(10) Pub. No.: US 2023/0073375 A1
(43) Pub. Date: Mar. 9, 2023

(54) AN IMAGE DETECTOR

(71) Applicant: PURE LUMINESCENCE TECHNOLOGIES OY, Turku (FI)

(72) Inventors: Mika LASTUSAARI, Turku (FI); Isabella NORRBO, Turku (FI); Sami VUORI, Paimio (FI); Hannah BYRON, Turku (FI)

(21) Appl. No.: 17/796,725

(22) PCT Filed: Nov. 11, 2020
(86) PCT No.: PCT/FI2020/050742

§ 371 Date: Aug. 1, 2022

(30) Foreign Application Priority Data
Jan. 30, 2020 (FI) 20205093

(51) Int. Cl. G01T 1/20; G03B 42/02

(52) U.S. Cl. CPC G01T 1/2014; G03B 42/02

(57) ABSTRACT

An image detector for a radiation-based imaging technique is disclosed. The image detector may comprise a detector material on a substrate. The detector material may be an optically active material represented by the following formula (I) (M′)₈ (M″M′″)₆O₂₄(X,X′)₂:M″″ Further is disclosed the use of the image detector and the use of the optically active material represented by the formula (I).

### TECHNICAL FIELD

The present disclosure relates to an image detector for a radiation-based imaging technique. The present disclosure further relates to the use of the image detector and to the use of an optically active material.

### BACKGROUND

Medical imaging is the technique and process of creating visual representations of the interior of a body for clinical analysis and medical intervention, as well as visual representation of the function of some organs or tissues (physiology). Medical imaging seeks to reveal internal structures hidden by the skin and bones, as well as to diagnose and treat disease. Medical imaging also establishes a database of normal anatomy and physiology to make it possible to identify abnormalities. Currently different imaging plates and systems including materials like Ba(F,Cl,Br,I)₂:Eu or CsI:Tl are used in medical imaging. The inventors have recognized the need to construct an image detector, comprising a non-toxic material as the detector material, to be used in various imaging applications such as in medical imaging but also in imaging carried out in the industry.

### SUMMARY

An image detector for a radiation-based imaging technique is disclosed. The image detector may comprise a detector material on a substrate. The detector material may be an optically active material represented by the following formula (I)

(M′)₈(M″M′″)₆O₂₄(X,X′)₂:M″″

wherein

M′ represents a monoatomic cation of an alkali metal selected from Group 1 of the IUPAC periodic table of the elements, or of an alkaline earth metal selected from Group 2 of the IUPAC periodic table of the elements, or any combination of such cations;

M″ represents a trivalent monoatomic cation of an element selected from Group 13 of the IUPAC periodic table of the elements, or of a transition element selected from any of Groups 3-12 of the IUPAC periodic table of the elements, or any combination of such cations;

M′″ represents a monoatomic cation of an element selected from Group 14 of the IUPAC periodic table of the elements, or of an element selected from any of Groups 13 and 15 of the IUPAC periodic table of the elements, or of Zn, or any combination of such cations;

X represents an anion of an element selected from Group 17 of the IUPAC periodic table of the elements, or any combination of such anions, or wherein X is absent;

X′ represents an anion of one or more elements selected from Group 16 of the IUPAC periodic table of the elements, or any combination of such anions, or wherein X′ is absent; and

M″″ represents a dopant cation of an element selected from rare earth metals of the IUPAC periodic table of the elements, or from transition metals of the IUPAC periodic table of the elements, or of Ba, Sr, Tl, Pb, or Bi, or any combination of such cations, or wherein M″″ is absent;

with the proviso that at least one of X and X′ is present

Further disclosed is the use of the image detector as disclosed in the current specification for point-of-care analysis. Further disclosed is the use of an optically active material represented by the formula (I) as disclosed in the current specification as a detector material in a radiation-based imaging technique.

### BRIEF DESCRIPTION OF THE DRAWINGS

The accompanying drawings, which are included to provide a further understanding of the embodiments and constitute a part of this specification, illustrate embodiments and together with the description help to explain the principles of the above. In the drawings:

FIG. 1 and FIG. 2 disclose test results of example 2;

FIG. 3 discloses the image produced in example 4;

FIG. 4a-4z disclose images example 5;

FIG. 5a-5p disclose images of example 6;

FIG. 6 disclose images of example 7; and

FIG. 7 discloses one embodiment of the image detector.

### DETAILED DESCRIPTION

The present disclosure relates to an image detector for a radiation-based imaging technique. The image detector may comprise a detector material on a substrate. The detector material may be an optically active material represented by the following formula (I)

(M′)₈(M″M″′)₆O₂₄(X,X′)₂:M″″

wherein

M′ represents a monoatomic cation of an alkali metal selected from Group 1 of the IUPAC periodic table of the elements, or of an alkaline earth metal selected from Group 2 of the IUPAC periodic table of the elements, or any combination of such cations;

M″ represents a trivalent monoatomic cation of an element selected from Group 13 of the IUPAC periodic table of the elements, or of a transition element selected from any of Groups 3-12 of the IUPAC periodic table of the elements, or any combination of such cations;

M′″ represents a monoatomic cation of an element selected from Group 14 of the IUPAC periodic table of the elements, or of an element selected from any of Groups 13 and 15 of the IUPAC periodic table of the elements, or of Zn, or any combination of such cations;

X represents an anion of an element selected from Group 17 of the IUPAC periodic table of the elements, or any combination of such anions, or wherein X is absent;

X′ represents an anion of one or more elements selected from Group 16 of the IUPAC periodic table of the elements, or any combination of such anions, or wherein X′ is absent; and

M″″ represents a dopant cation of an element selected from rare earth metals of the IUPAC periodic table of the elements, or from transition metals of the IUPAC periodic table of the elements, or of Ba, Sr, Tl, Pb, or Bi, or any combination of such cations, or wherein M″″ is absent;

with the proviso that at least one of X and X′ is present.

Further the present disclosure relates to the use of the image detector as disclosed in the current specification for point-of-care analysis. Further the present disclosure relates to the use of an optically active material represented by the formula (I) as disclosed in the current specification as a detector material in an image detector for a radiation-based imaging technique.

In one embodiment, the image detector is a reusable image detector. The image detector has the added utility of one being able to reuse the same image detector one or several times.

The image detector is any suitable image detector capable of gathering the energy from the radiation that it is exposed to in the optically active material thereof. The image detector may be an imaging plate, an imaging sensor, or an imaging cell.

In one embodiment, the radiation used in the radiation-based imaging technique is a predetermined type of particle radiation. In one embodiment, the particle radiation is alfa radiation, beta radiation, neutron radiation, or any combination thereof.

In one embodiment, the radiation used in the radiation-based imaging technique is electromagnetic radiation having a wavelength of above 0 nm to 590 nm, or above 0 nm to 560 nm, or above 0 nm to 500 nm, or above 0 nm to 400 nm, or above 0 nm to 300 nm, or 0.000001-590 nm, or 0.000001-560 nm, or 0.000001 500 nm, or 10-590 nm, or 10-560 nm, or 10-500 nm, or 0.000001-400 nm, or 0.000001-300 nm, or 0.000001-10 nm, or 10-400 nm, or 10-300 nm, or 0.01-10 nm.

In one embodiment, the radiation used in the radiation-based imaging technique is ultraviolet radiation, X-radiation, gamma radiation, or any combination thereof. In one embodiment, the radiation used in the radiation-based imaging technique is ultraviolet radiation. In one embodiment, the radiation used in the radiation-based imaging technique is X-radiation. In one embodiment, the radiation used in the radiation-based imaging technique is gamma radiation.

In one embodiment, the radiation-based imaging technique is an X-ray-based imaging technique, a UV-radiation-based imaging technique, or a gamma-radiation-based imaging technique.

Ultraviolet light is electromagnetic radiation with a wavelength from 10 nm (30 PHz) to 400 nm (750 THz). The electromagnetic spectrum of ultraviolet radiation (UVR) can be subdivided into a number of ranges recommended by the ISO standard ISO-21348, including ultraviolet A (UVA), ultraviolet B (UVB), ultraviolet C (UVC). The wavelength of UVA is generally considered to be 315-400 nm, the wavelength of UVB is generally considered to be 280-320 and the wavelength of UVC is generally considered to be 100-290 nm.

Gamma radiation is electromagnetic radiation with a wavelength from 0.000001 nm to 0.01 nm.

In one embodiment, the X-ray-based imaging technique is X-ray imaging, computed radiography (CR), digital radiography (DR), or computed tomography (CT).

X-radiation is electromagnetic radiation with a wavelength from 0.01 nm to 10 nm. X-rays are electromagnetic radiation that differentially penetrates structures within e.g. a body or a tissue and creates images of these structures on an image detector. Thus, X-ray based imaging may create pictures of the inside of e.g. the body. The images may show the parts of the body in different shades of black and white. This is because different tissues absorb different amounts of radiation. Thus, when imaging with X-rays, an X-ray beam produced by a so-called X-ray tube passes through the body. On its way through the body, parts of the energy of the X-ray beam are absorbed. This process is described as attenuation of the X-ray beam. On the opposite side of the body, the image detector captures the X-rays that are not absorbed, resulting in a clinical image. In conventional radiography, i.e. X-ray imaging, one 2D image is produced. In computed tomography (CT), the tube and the image detector are both rotating around the body during the examination so that multiple images can be acquired, resulting in a 3D visualization.

In computed radiography, when image detectors are exposed to X-rays, the energy of the incoming radiation is stored or retained in the optically active material. A scanner may then be used to read out the latent image from the image detector by stimulating it with a laser beam. When stimulated, the plate emits light with intensity proportional to the amount of radiation received during the exposure. The light may then be detected by a highly sensitive analog device known as a photomultiplier (PMT) and converted to a digital signal using an analog-to-digital converter (ADC). The generated digital X-ray image may then be viewed on a computer monitor and evaluated.

Digital radiography uses X-ray-sensitive image detectors that directly capture data during the patient examination, immediately transferring it to a computer system without the use of an intermediate cassette as is the case with computed radiography (CR). The optically active material in the image detector converts the X-ray exposed thereon to visible light which may then be translated into digital data.

The above imaging systems or processes are based on the idea that X-radiation is being exposed to the image detector comprising the optically active material as the detector material.

The inventors surprisingly found out that the optically active material represented by formula (I) as described in the current specification, may be used as a detector material in imaging applications. The optically active material as disclosed in the current specification has the added utility of being able to retain radiation such as X-radiation exposed thereon.

The optically active material has the added utility of being able to change color under the exposure to radiation. The intensity of the color is dependent on the amount of radiation, such as X-radiation or ultraviolet radiation, that reaches the detector material. The color change of the detector material may be based on photochromism. X-rays may induce color centers in the detector material. The more X-rays that hit the material the more color centers are formed and thus a deeper color is obtained. In one embodiment, the optically active material is a photochromic material.

In one embodiment, the detector material is configured to retain radiation, e.g. X-radiation, exposed thereon for a predetermined period of time. In one embodiment, the detector material is configured to release the retained radiation, e.g. X-radiation, as visible light when being subjected to heat treatment and/or optical stimulation.

When in use the image detector with the optically active material as detector material may be exposed to radiation, e.g. X-radiation, for a predetermined period of time, such as for 0.01 seconds-10 minutes, or 0.1 seconds-5 minutes, or 5 seconds-1 minute. The time the optically active material is allowed to be exposed to the radiation may depend on the application where the optically active material is used and thus on the amount of radiation to which the optically active material is to be exposed to.

The irradiated radiation, e.g. X-radiation, may be retained in the optically active material of the image detector for a predetermined period of time. Then the optically active material may be subjected to e.g. heating and/or optical stimulation to release the retained radiation from the optically active material. In one embodiment, the predetermined period of time is at least 1 minute, or at least 2 minutes, or at least 5 minutes, or at least 10 minutes, or at least 15 minutes, or at least 0.5 hour, or at least 1 hour, or at least 2 hours, or at least 5 hours, or at least 6 hours, or at least 8 hours, or at least 12 hours, or at least 18 hours, or at least 24 hours, or at least one week, or at least one month. In one embodiment, the predetermined period of time is at most 3 months, or at most one month, or at most one week, or at most 24 hours. In one embodiment, the predetermined period of time is 1 minute-3 months, or 10 minutes-one month, or 0.5 h-one week. In one embodiment, said predetermined period of time is 0.5 h-3 months.

The optically active material as described in current specification has the ability to retain radiation energy, i.e. the optically active material is able to trap therein the radiation that it is exposed to. The retained radiation may be released from the optically active material later at a predetermined point of time. The optically active material may emit visible light as a result of changing, e.g. increasing or decreasing, the temperature thereof and/or as a result of optical stimulation.

Optical stimulation of the optically active material may comprise subjecting the optically active material to electromagnetic radiation having a wavelength of 310-1400 nm. In one embodiment, the optical stimulation of the optically active material comprises subjecting the optically active material to visible light, ultraviolet radiation and/or to near infrared radiation. The optical stimulation of the optically active material may be carried out by using a laser, a light emitting diode (LED), an organic light-emitting diode (OLED), an active-matrix organic light emitting diode (AMOLED), an incandescent lamp, a halogen lamp, any other optical stimulation luminescence light source, or any combination thereof.

The optically active material described in the current specification, as a result of being subjected to radiation, e.g. X-radiation, has the added utility of showing color intensity, which is proportional with the dose of the radiation that is has been exposed to.

In one embodiment, the image detector is used in diagnostics. The image detector comprising the optically active material described in the current specification as the detector material can be used in diagnosing a sample received from human or animal body or in diagnosing the human or animal body directly. In one embodiment, the sample is selected from a group consisting of a body fluid, a tooth, a bone, and a tissue. In one embodiment, the sample comprises blood, skin, tissue and/or cells. The image detector comprising the optically active material described in this specification may be used in in vivo imaging or in in vivo diagnostics. In one embodiment, the imaging is medical imaging. The image plate described in the current specification may be used in detection technology.

In one embodiment, the image detector as described in the current specification is used in point-of-care testing. Point-of-care testing (POCT), also called bedside testing, may be defined as medical diagnostic testing at or near the point of care, i.e. at the time and place of patient care. This is contrary to the situation wherein testing is wholly or mostly confined to a medical laboratory, which entails sending off a specimen away from the point of care and then waiting e.g. hours or days to learn the results.

In one embodiment, the image detector as described in the current specification is used in imaging carried out in the industry. The image detector as described in the current specification may be used in non-destructive testing. The image detector as described in the current specification may be used e.g. to imaging welding.

In one embodiment, the optically active material is a synthetic material. In one embodiment, the optically active material is synthetically prepared.

In this specification, unless otherwise stated, the expression “monoatomic ion” should be understood as an ion consisting of a single atom. If an ion contains more than one atom, even if these atoms are of the same element, it is to be understood as a polyatomic ion. Thus, in this specification, unless otherwise stated, the expression “monoatomic cation” should be understood as a cation consisting of a single atom. Hackmanite, which is a variety of sodalite material, is natural mineral having the chemical formula of Na₈Al₆Si₆O₂₄(Cl,S)₂. A synthetic hackmanite-based material can be prepared.

The optically active material represented by formula (I), as a result of being exposed to X-radiation, has the added utility of emitting white light. The expression “luminescent” may in this specification, unless otherwise stated, refer to the property of the material to being able to emit light without being heated.

In one embodiment, M′ represents a monoatomic cation of an alkali metal selected from a group consisting of Na, Li, K, Rb, Cs, and Fr, or any combination of such cations. In one embodiment, M′ represents a monoatomic cation of an alkali metal selected from a group consisting of Li, K, Rb, Cs, and Fr, or any combination of such cations.

In one embodiment, M′ represents a monoatomic cation of an alkali metal selected from Group 1 of the IUPAC periodic table of the elements, or of an alkaline earth metal selected from Group 2 of the IUPAC periodic table of the elements, or any combination of such cations; with the proviso that M′ does not represent the monoatomic cation of Na alone. In one embodiment, M′ does not represent the monoatomic cation of Na alone.

In one embodiment, M′ represents a monoatomic cation of an alkaline earth metal selected from a group consisting of Be, Mg, Ca, Sr, Ba, Ra, or any combination of such cations. In one embodiment, M′ represents a monoatomic cation of Ca.

In one embodiment, M′ represents a monoatomic cation of a metal selected from a group consisting of Li, K, Rb, Cs, Fr, Be, Mg, Ca, Sr, Ba, Ra, or any combination of such cations.

In one embodiment, M′ represents a combination of at least two monoatomic cations of different metals, wherein at least one metal is selected from Group 1 of the IUPAC periodic table of the elements and at least one metal is selected from Group 2 of the IUPAC periodic table of the elements.

In one embodiment, M′ represents a combination of at least two monoatomic cations of different alkali metals selected from Group 1 of the IUPAC periodic table of the elements. In one embodiment, M′ represents a combination of at least two monoatomic cations of different alkaline earth metals selected from Group 2 of the IUPAC periodic table of the elements.

In one embodiment, M′ represents a combination of at least two monoatomic cations of different alkali metals selected from Group 1 of the IUPAC periodic table of the elements and/or alkaline earth metals selected from Group 2 of the IUPAC periodic table of elements, and wherein the combination comprises at most 98 mol-%, at most 95 mol-%, at most 90 mol-%, at most 85 mol-%, at most 80 mol-%, at most 70 mol-%, at most 60 mol-%, at most 50 mol-%, at most 40 mol-% of the monoatomic cation of Na, or at most 30 mol-% of the monoatomic cation of Na, or at most 20 mol-% of the monoatomic cation of Na.

In one embodiment, M′ represents a combination of at least two monoatomic cations of different alkali metals selected from Group 1 of the IUPAC periodic table of the elements and/or alkaline earth metals selected from Group 2 of the IUPAC periodic table of elements, wherein the combination comprises 0-98 mol-%, or 0-95 mol-%, or 0-90 mol-%, or 0-85 mol-%, or 0-80 mol-%, or 0-70 mol-%, of the monoatomic cation of Na.

In one embodiment, M′ represents a monoatomic cation of Li. In one embodiment, M′ represents a monoatomic cation of K. In one embodiment, M′ represents a monoatomic cation of Rb. In one embodiment, M′ represents a monoatomic cation of Cs. In one embodiment, M′ represents a monoatomic cation of Fr. In one embodiment, M′ represents a monoatomic cation of Ca.

In one embodiment, M″ represents a trivalent monoatomic cation of a metal selected from a group consisting of Al and Ga, or a combination of such cations.

In one embodiment, M″ represents a trivalent monoatomic cation of B.

In one embodiment, M″ represents a trivalent monoatomic cation of a transition element selected from any of Period 4 of the IUPAC periodic table of the elements, or any combination of such cations.

In one embodiment, M″ represents a trivalent monoatomic cation of an element selected from a group consisting of Cr, Mn, Fe, Co, Ni, and Zn, or any combination of such cations.

In one embodiment, M′″ represents a monoatomic cation of an element selected from a group consisting of Si, Ge, Al, Ga, N, P, and As, or any combination of such cations.

In one embodiment, M′″ represents a monoatomic cation of an element selected from a group consisting of Si and Ge, or a combination of such cations.

In one embodiment, M′″ represents a monoatomic cation of an element selected from a group consisting of Al, Ga, N, P, and As, or any combination of such cations.

In one embodiment, M′″ represents a monoatomic cation of an element selected from a group consisting of Al and Ga, or a combination of such cations.

In one embodiment, M′″ represents a monoatomic cation of an element selected from a group consisting of N, P, and As, or any combination of such cations.

In one embodiment, M′″ represents a monoatomic cation of Zn.

In one embodiment, X represents an anion of an element selected from a group consisting of F, Cl, Br, I, and At, or any combination of such anions. In one embodiment, X represents an anion of an element selected from a group consisting of F, Cl, Br, and I, or any combination of such anions. In one embodiment, X is absent.

In one embodiment, X′ represents an anion of an element selected from a group consisting of O, S, Se, and Te, or any combination of such anions. In one embodiment, X′ represents an anion of one or more elements selected from a group consisting of O, S, Se, and Te, or any combination of such anions. In one embodiment, X′ represents a monoatomic or a polyatomic anion of one or more elements selected from a group consisting of O, S, Se, and Te, or any combination of such anions. In one embodiment, X′ represents an anion of S. In one embodiment, X′ is (SO4)²⁻. In one embodiment X′ is absent.

The proviso that at least one of X and X′ is present should in this specification, unless otherwise stated, be understood such that either X or X′ is present, or such that both X and X′ are present.

In one embodiment, the optically active material is doped with at least one transition metal ion. In one embodiment, the optically active material is represented by formula (I), wherein M″″ represents a cation of an element selected from transition metals of the IUPAC periodic table of the elements, or of Ba, Sr, Tl, Pb, or Bi, or any combination of such cations. In one embodiment, M″″ represents a cation of an element selected from transition metals of the f-block of the IUPAC periodic table of the elements. In one embodiment, M″″ represents a cation of an element selected from transition metals of the d-block of the IUPAC periodic table of the elements. In one embodiment, M″″ represents a cation of an element selected from a group consisting of Ti, V, Cr, Mn, Fe, Co, Ni, Cu, Ag, W, and Zn, or any combination of such cations. In one embodiment, M″″ represents a cation of Ti. In one embodiment, M″″ represents a dopant cation of an element selected from rare earth metals of the IUPAC periodic table of the elements. In one embodiment, M″″ represents a cation of an element selected from a group consisting of Yb, Er, Tb, and Eu, or any combination of such cations. In one embodiment, M″″ represents a combination of two or more dopant cations.

In one embodiment, the optically active material is represented by formula (I), wherein M″″ is absent. In this embodiment, the optically active material is not doped.

In one embodiment, the optically active material represented by the formula (I) comprises M″″ in an amount of 0.001-10 mol-%, or 0.001-5 mol-%, or 0.1-5 mol-% based on the total amount of the optically active material.

In one embodiment, the optically active material is selected from a group consisting of:

(LiₓNa_{1-x-y-z}K_{y}Rb_{z})₈(Al,Ga)₆Si₆O₂₄(Cl,S)₂:Ti,

(LiₓNa_{1-x-y-z}K_{y}Rb_{z})₈(Al,Cr)₆Si₆O₂₄(Cl,S)₂:Ti

(LiₓNa_{1-x-yz}K_{y}Rb_{z})₈(Al,Mn)₆Si₆O₂₄(Cl,S)₂:Ti,

(LiₓNa_{1-x-y-z}K_{y}Rb_{z})₈(Al,Fe)₆Si₆O₂₄(Cl,S)₂:Ti,

(LiₓNa_{1-x-y-z}K_{y}Rb_{z})₈(Al,Co)₆Si₆O₂₄(Cl,S)₂:Ti,

(LiₓNa_{1-x-y-z}K_{y}Rb_{z})₈(Al,Ni)₆Si₆O₂₄(Cl,S)₂:Ti,

(LiₓNa_{1-x-y-z}K_{y}Rb_{z})₈(Al,Cu)₆Si₆O₂₄(Cl,S)₂:Ti,

(LiₓNa_{1-x-y-z}K_{y}Rb_{z})₈(Al,B)₆Si₆O₂₄(Cl,S)₂:Ti,

(LiₓNa_{1-x-y-z}K_{y}Rb_{z})₈Mn₆Si₆O₂₄(Cl,S)₂:Ti,

(LiₓNa_{1-x-y-z}K_{y}Rb_{z})₈Cr₆Si₆O₂₄(Cl,S)₂:Ti,

(LiₓNa_{1-x-y-z}K_{y}Rb_{z})₈Fe₆Si₆O₂₄(Cl,S)₂:Ti,

(LiₓNa_{1-x-y-z}K_{y}Rb_{z})₈Co₆Si₆O₂₄(Cl,S)₂:Ti,

(LiₓNa_{1-x-y-z}K_{y}Rb_{z})₈Ni₆Si₆O₂₄(Cl,S)₂:Ti,

(LiₓNa_{1-x-y-z}K_{y}Rb_{z})₈Cu₆Si₆O₂₄(Cl,S)₂:Ti

(LiₓNa_{1-x-y-z}K_{y}Rb_{z})₈B₆Si₆O₂₄(Cl,S)₂:Ti,

(LiₓNa_{1-x-y-z}K_{y}Rb_{z})₈Ga₆Si₆O₂₄(Cl,S)₂:Ti,

(LiₓNa_{1-x-y-z}K_{y}Rb_{z})₈Al₆(Si,Zn)₆O₂₄(Cl,S)₂:Ti,

(LiₓNa_{1-x-y-z}K_{y}Rb_{z})₈Al₆(Si,Ge)₆O₂₄(Cl,S)₂:Ti,

(LiₓNa_{1-x-y-z}K_{y}Rb_{z})₈Al₆Zn₆O₂₄(Cl,S)₂:Ti,

(LiₓNa_{1-x-y-z}K_{y}Rb_{z})₈Al₆Ge₆O₂₄(Cl,S)₂:Ti,

(LiₓNa_{1-x-y-z}K_{y}Rb_{z})₈Al₆(Ga,Si,N)₆O₂₄(Cl,S)₂:Ti,

(LiₓNa_{1-x-y-z}K_{y}Rb_{z})₈Al₆(Ga,Si,As)₆O₂₄(Cl,S)₂:Ti,

(LiₓNa_{1-x-y-z}K_{y}Rb_{z})₈Al₆(Ga,N)₆O₂₄(Cl,S)₂:Ti,

(LiₓNa_{1-x-y-z}K_{y}Rb_{z})₈Al₆(Ga,As)₆O₂₄(Cl,S)₂:Ti,

(LiₓNa_{1-x-y-z}K_{y}Rb_{z})₈(Al,Ga)₆Ge₆O₂₄(Cl,S)₂:Ti

(LiₓNa_{1-x-y-z}K_{y}Rb_{z})₈(Al,Cr)₆Ge₆O₂₄(Cl,S)₂:Ti,

(LiₓNa_{1-x-y-z}K_{y}Rb_{z})₈(Al,Mn)₆Ge₆O₂₄(Cl,S)₂:Ti,

(LiₓNa_{1-x-y-z}K_{y}Rb_{z})₈(Al,Fe)₆Ge₆O₂₄(Cl,S)₂:Ti,

(LiₓNa_{1-x-y-z}K_{y}Rb_{z})₈(Al,Co)₆Ge₆O₂₄(Cl,S)₂:Ti,

(LiₓNa_{1-x-y-z}K_{y}Rb_{z})₈(Al,Ni)₆Ge₆O₂₄(Cl,S)₂:Ti

(LiₓNa_{1-x-y-z}K_{y}Rb_{z})₈(Al,Cu)₆Ge₆O₂₄(Cl,S)₂:Ti,

(LiₓNa_{1-x-y-z}K_{y}Rb_{z})₈(Al,B)₆Ge₆O₂₄(Cl,S)₂:Ti,

(LiₓNa_{1-x-y-z}K_{y}Rb_{z})₈Mn₆Ge₆O₂₄(Cl,S)₂:Ti,

(LiₓNa_{1-x-y-z}K_{y}Rb_{z})₈Cr₆Ge₆O₂₄(Cl,S)₂:Ti,

(LiₓNa_{1-x-y-z}K_{y}Rb_{z})₈Fe₆Ge₆O₂₄(Cl,S)₂:Ti,

(LiₓNa_{1-x-y-z}K_{y}Rb_{z})₈Co₆Ge₆O₂₄(Cl,S)₂:Ti,

(LiₓNa_{1-x-y-z}K_{y}Rb_{z})₈Ni₆Ge₆O₂₄(Cl,S)₂:Ti,

(LiₓNa_{1-x-y-z}K_{y}Rb_{z})₈Cu₆Ge₆O₂₄(Cl,S)₂:Ti,

(LiₓNa_{1-x-y-z}K_{y}Rb_{z})₈B₆Ge₆O₂₄(Cl,S)₂:Ti, and

(LiₓNa_{1-x-y-z}K_{y}Rb_{z})₈Ga₆Ge₆O₂₄(Cl,S)₂:Ti,

wherein

x+y+z≤1, and

x≥0, y≥0, z≥0.

The optically active material may be synthesized by a reaction according to Norrbo et al. (Norrbo, I.; Gluchowski, P.; Paturi, P.; Sinkkonen, J.; Lastusaari, M., Persistent Luminescence of Tenebrescent Na₈Al₆Si₆O₂₄(Cl,S)₂: Multifunctional Optical Markers. Inorg. Chem. 2015, 54, 7717-7724), which reference is based on Armstrong & Weller (Armstrong, J. A.; Weller, J. A. Structural Observation of Photochromism. Chem. Commun. 2006, 1094-1096). As an example, stoichiometric amounts of Zeolite A and Na₂SO₄ as well as LiCl, NaCl, KCl and/or RbCl can be used as the starting materials. The at least one dopant may be added as an oxide, such as TiO₂, a chloride, a sulfide, a bromide, or a nitrate. The material can be prepared as follows: Zeolite A may first be dried at 500° C. for 1 h. The initial mixture may then be heated at 850° C. in air for e.g. 2 h, 5 h, 12 h, 24 h, 36 h, 48 h, or 72 h. The product may then be freely cooled down to room temperature and ground. Finally, the product may be re-heated at 850° C. for 2 h under a flowing 12% H₂+88% N₂ atmosphere. If needed, the as-prepared materials may be washed with water to remove any excess LiCl/NaCl/KCl/RbCl impurities. The purity can be verified with an X-ray powder diffraction measurement.

The image detector may be produced following any known technique by using the optically active material as described in the current specification. Tape casting, also known as knife coating or doctor blading, may be used for producing the image detector. Tape casting is a process where a thin sheet of ceramic or metal particle suspension fluid is cast on a substrate. The fluid may contain volatile nonaqueous solvents, a dispersant, (a) binder(s) and the dry matter, i.e. the optically active material. The process may comprise preparing the suspension and applying it onto a surface of a substrate. The binder may create a polymer network around the dry matter particles, while the plasticizer may function as a softening agent for the binder. When combining these substances, the tape may become resistant against cracking and flaking off when bent. The dispersant may be used to deaggregate the particles and homogenize the suspension. The image detector comprising the optically active material may be prepared following the description given in e.g. Abhinay et al., Tape casting and electrical characterization of 0.5Ba(Zr_{0.2}Ti_{0.8})O₃-0.5(Ba_{0.7}Ca_{0.3})TiO₃ (BZT-0.5BCT) piezoelectric substrate; Journal of the European Ceramic Society 36 (2016) 3125-3137.

The substrate of the image detector may comprise or consist of glass or polymer. The substrate may comprise or consist of a glass layer or a polymer layer. The substrate may comprise (a) further layer(s). The substrate may comprise an attachment layer, such as a printing paper, and/or a base layer, such as a cardboard layer, or any other layer(s) where desired or needed. The image detector may comprise further layers and/or components.

The image detector disclosed in the current specification has the added utility of enabling the use of the optically active material represented by formula (I) as described in the current specification as a detector material for imaging purposes. The image detector disclosed in the current specification has the added utility of making use of an optically active material being non-toxic and non-expensive compared to currently used materials such as Ba(F,Cl,Br,I)₂:Eu and CsI:Ti. The image detector as disclosed in the current specification has the added utility of being reusable and recyclable. Further, the image detector as disclosed in the current specification can be used for point-of-care analysis without the need of complicated analysis systems.

It will be understood that the benefits and advantages described above may relate to one embodiment or may relate to several embodiments. The embodiments are not limited to those that solve any or all of the stated problems or those that have any or all of the stated benefits and advantages.

The embodiments of the invention described hereinbefore may be used in any combination with each other. Several of the embodiments may be combined together to form a further embodiment of the invention. An image detector, or a use, to which the current specification is related, may comprise at least one of the embodiments described hereinbefore.

### EXAMPLES

Reference will now be made in detail to various embodiments, examples of which are illustrated in the accompanying drawings.

The description below discloses some embodiments in such a detail that a person skilled in the art is able to utilize the embodiments based on the disclosure. Not all steps or features of the embodiments are discussed in detail, as many of the steps or features will be obvious for the person skilled in the art based on this specification.

The enclosed FIG. 7 discloses an example of an embodiment of the image detector. FIG. 7 discloses an image detector 1 that comprises the detector material 2 on the substrate 3. The detector material is the optically active material represented by formula (I) as described in the current specification. In the embodiment of FIG. 7, the substrate 3 comprises a casting layer 3a, an attachment layer 3b, and a base layer 3c. The casting layer 3a may be formed of a polymer, such as polyester, the attachment layer 3b may be formed of a printing paper, and the base layer 3c may be formed of cardboard. The thicknesses of the different layers may vary but as an example only the casting layer 3a may have a thickness of about 100 μm, the attachment layer 3b may have a thickness of about 65 μm, and the base layer 3c may have a thickness of about 250 μm. The layer of the detector material 2 may have a thickness of about 100 μm.

### Example 1—Preparing Materials

The materials in the below table were prepared using the following starting materials:

| Material to be | | Heating |
|---|---|---|
| prepared | Starting materials | time (h) |
| LiNa₆K(AlSiO₄)₆ | Zeolite A, LiCl, KCl, | 5 |
| (Cl, S)₂ | Na₂SO₄ | |
| Na₈(AlSiO₄)₆ | Zeolite A, NaCl, Na₂SO₄ | 48 |
| (Cl, S)₂ | | |
| (Na, Ca)₈(AlSi | Zeolite A, NaCl, CaCl₂, | 48 |
| O₄)₆(Cl, S)₂ | Na₂SO₄ | |
| Na₈(AlSiO₄)₆ | ZeoliteA, NaCl, | 2 |
| (Cl, S)₂:W | Na₂SO₄, WS₂ | |
| LiNa₇(AlSiO₄)₆ | Zeolite A, NaBr, LiBr, | 5 |
| (Br, S)₂ | Na₂SO₄ | |
| Na₈(AlSiO₄)₆ | Zeolite A, NaCl, | 48 |
| (Cl, S)₂:Os | Na₂SO₄, OSCl₄ | |
| Na₈(AlSiO₄)₆ | Zeolite A, NaBr, Na₂SO₄ | 48 |
| (Br, S)₂ | | |

The materials were prepared in the following manner: the starting materials were mixed together in stoichiometric ratios. The mixture was heated at 850° C. in air for the time periods indicated in the above table. The product was freely cooled down to room temperature and ground. Finally, the product was re-heated at 850° C. for 2 h under a flowing 12% H₂+88% N₂ atmosphere.

### Example 2—Testing of the Samples of the Materials of Example 1

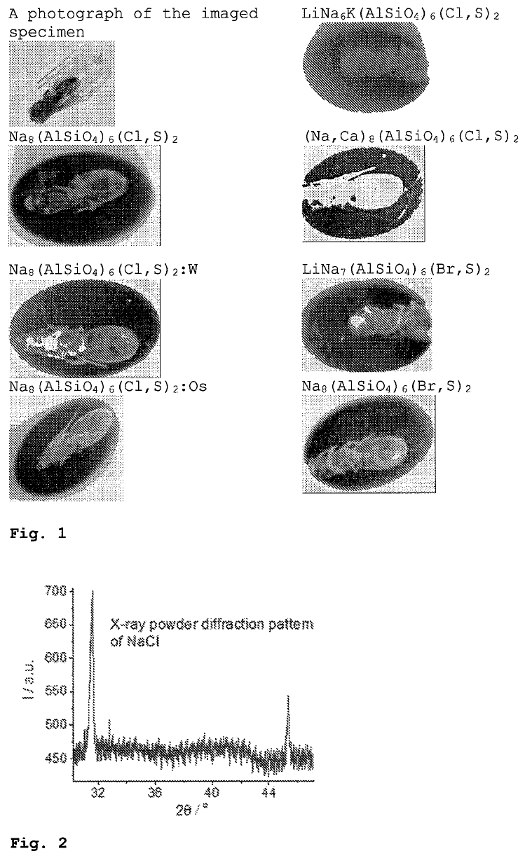

Each of the samples were subjected to X-ray imaging. For the X-ray imaging, the samples of the material were attached to the surface of a 50 μm thick polymer film with tape casting technique following the description given in: Abhinay et al., Tape casting and electrical characterization of 0.5Ba(Zr_{0.2}Ti_{0.8})O₃-0.5(Ba_{0.7}Ca_{0.3})TiO₃ (EZT-0.5BCT) piezoelectric substrate; Journal of the European Ceramic Society 36 (2016) 3125-3137. The obtained films were glued to cardboard plates. The images were created using the X-ray beam of an X-ray fluorescence spectrometer (Ag tube; E˜20 keV) and a dead winged ant as the specimen. The image data was read with an unmodified intraoral X-ray image reader Durr Dental VistaScan. That device operates with a 635 nm stimulation. Photographs of the imaged specimen are presented in FIG. 1.

Further, each of the samples were subjected to X-ray diffraction. The difference between the X-ray imaging and X-ray diffraction applications is that the imaging creates a 2D image whereas the diffraction represents a line scan. For the X-ray diffraction image plate, a sample of the material was attached to the surface of a 50 μm thick polymer film with the above tape casting technique. The obtained film was attached inside an otherwise unmodified Huber G670 detector. The X-radiation used was copper K alpha 1 (E=8.0 keV) and the specimen was NaCl powder. The G670 detector uses a 620 nm stimulation to read data from the image detector. FIG. 2 represents an example X-ray diffraction pattern obtained with a sample of material as the detector material, i.e. Na₈(AlSiO₄)₆(Br,S)₂. From the graph it can be seen that it is possible to use the optically active material as detector material in a commercial X-ray powder diffraction detector (Huber G670) operating with the OSL/PSL principle, i.e an X-ray powder diffraction pattern can be obtained by using the optically active material as described in the current specification.

### Example 3—Preparing Different Materials

Following the general description presented in example 1, the following materials were prepared by using the following starting materials:

| Material to be prepared | Starting materials | Heating time (h) |
|---|---|---|
| (Li, Na, K, Rb)₈(AlSi)₆ | Zeolite A, LiCl, | 48 |
| O₂₄(Cl, S)₂:Ti | NaCl, KCl, RbCl, | |
| | Na₂SO₄, TiO₂ | |
| (Li, Na, K, Rb)₈(AlSi)₆ | Zeolite A, LiCl, | 48 |
| O₂₄(Cl, S)₂:Ti, Eu | NaCl, KCl, RbCl, | |
| | Na₂SO₄, TiO₂, Eu₂O₃ | |
| (Li, Na, K, Rb)₈(AlSi)₆ | Zeolite A, LiCl, | 48 |
| O₂₄(Cl, S)_{2:}Ti, Bi | NaCl, KCl, RbCl, | |
| | Na₂SO₄, TiO₂, Bi₂O₃ | |
| (Li, Na, K, Rb)₈(AlSi)₆ | Zeolite A, LiCl, | 48 |
| O₂₄(Cl, S)_{2:}Ti, Yb, Er | NaCl, KCl, RbCl, | |
| | Na₂SO₄, TiO₂, Yb₂O₃, | |
| | Er₂O₃ | |
| (Li, Na, K, Rb)₈(AlSi)₆ | Zeolite A, LiCl, | 48 |
| O₂₄(Cl, S)_{2:}Ti, Cu | NaCl, KCl, RbCl, | |
| | Na₂SO₄, TiO₂, CuO | |
| (Li, Na, K, Rb)₈(AlSi)₆ | Zeolite A, LiCl, | 48 |
| O₂₄(Cl, S)₂:Ti, Mn | NaCl, KCl, RbCl, | |
| | Na₂SO₄, TiO₂, MnO | |
| (Li, Na, K, Rb)₈(Al, Ga)₆ | Zeolite A, LiCl, | 48 |
| Si₆O₂₄(Cl, S)₂:Ti | NaCl, KCl, RbCl, | |
| | Ga₂O₃, Na₂SO₄, TiO₂ | |
| (Li, Na, K, Rb)₈(Al, Cr)₆ | Zeolite A, LiCl, | 48 |
| Si₆O₂₄(Cl, S)₂:Ti | NaCl, KCl, RbCl, | |
| | Cr₂O₃, Na₂SO₄, TiO₂ | |
| (Li, Na, K, Rb)₈(Al, Mn)₆ | Zeolite A, LiCl, | 48 |
| Si₆O₂₄(Cl, S)₂:Ti | NaCl, KCl, RbCl, | |
| | MnO Na₂SO₄, TiO₂ | |
| (Li, Na, K, Rb)₈(Al, Fe)₆ | Zeolite A, LiCl, | 48 |
| Si₆O₂₄(Cl, S)₂:Ti | NaCl, KCl, RbCl, | |
| | FeO, Na₂SO₄, TiO₂ | |
| (Li, Na, K, Rb)₈(Al, Co)₆ | Zeolite A, LiCl, | 48 |
| Si₆O₂₄(Cl, S)₂:Ti | NaCl, KCl, RbCl, | |
| | CoO, Na₂SO₄, TiO₂ | |
| (Li, Na, K, Rb)₈(Al, Ni)₆ | Zeolite A, LiCl, | 48 |
| Si₆O₂₄(Cl, S)₂:Ti | NaCl, KCl, RbCl, | |
| | NiO, Na₂SO₄, TiO₂ | |
| (Li, Na, K, Rb)₈(Al, Cu)₆ | Zeolite A, LiCl, | 48 |
| Si₆O₂₄(Cl, S)₂:Ti | NaCl, KCl, RbCl, | |
| | CuO, Na₂SO₄, TiO₂ | |
| (Li, Na, K, Rb)₈(Al, B)₆ | Zeolite A, LiCl, | 48 |
| Si₆O₂₄(Cl, S)₂:Ti | NaCl, KCl, RbCl, | |
| | B₂O₃, Na₂SO₄, TiO₂ | |
| (Li, Na, K, Rb)Al₆(Si, | Zeolite A, LiCl, | 48 |
| Zn)₆O₂₄(Cl, S)₂:Ti | NaCl, KCl, RbCl, | |
| | ZnO, Na₂SO₄, TiO₂ | |
| (Li, Na, K, Rb)₈Al₆(Si, | Zeolite A, LiCl, | 48 |
| Ge)₆O₂₄(Cl, S)₂:Ti | NaCl, KCl, RbCl, | |
| | GeO₂, Na₂SO₄, TiO₂ | |
| (Li, Na, K, Rb)₈Al₆(Ga, | Zeolite A, LiCl, | 48 |
| Si)₆O₂₄(Cl, S)_{2:}Ti | NaCl, KCl, RbCl, | |
| | Ga₂O₃, Na₂SO₄, TiO₂ | |
| (Li, Na, K, Rb)₈Al₆(Si, | Zeolite A, LiCl, | 48 |
| As)₆O₂₄(Cl, S)₂:Ti | NaCl, KCl, RbCl, | |
| | AS₂O₃, Na₂SO₄, TiO₂ | |
| (Li, Na, K, Rb)₈Al₆(Si, | Zeolite A, LiCl, | 48 |
| N)₆O₂₄(Cl, S)_{2:}Ti | NaCl, KCl, RbCl, | |
| | NO, Na₂SO₄, TiO₂ | |
| (Li, Na, K, Rb)₈(AlSi)₆ | Zeolite A, LiCl, | 48 |
| O₂₄(Cl, Br, S)_{2:}Ti | NaCl, KCl, RbCl, | |
| | NaBr, Na₂SO₄, TiO₂, | |
| (Li, Na, K, Rb)₈(AlSi)₆ | Zeolite A, LiCl, | 48 |
| O₂₄(Cl, F, S)_{2:}Ti | NaCl, KCl, RbCl, | |
| | NaF, Na₂SO₄, TiO₂ | |
| LiNa₆(AlSiO₄)₆(Cl, S)₂ | Zeolite A, LiCl, | 48 |
| | Na₂SO₄ | |
| Li₂Na₆(AlSiO₄)₆(Cl, | Zeolite A, LiCl, | 48 |
| S)₂:Ti | | |
| Li₂Na₆(AlSiO₄)₆(Br, | Zeolite A, LiBr, | 48 |
| S)₂ | Na₂SO₄ | |
| Li₂Na₆(AlSiO₄)₆(Br, | Zeolite A, LiBr, | 48 |
| S)₂:Ti | Na₂SO₄, TiO₂ | |
| Na₈(AlSiO₄)₆(Cl, S)₂ | Zeolite A, NaCl, | 48 |
| | Na₂SO₄ | |
| Na₈(AlSiO₄)₆(Br, S)₂ | Zeolite A, NaBr, | 48 |
| | Na₂SO₄ | |
| Na₈(AlSiO₄)₆(Br, S)₂:Ti | Zeolite A, NaBr, | 48 |
| | Na₂SO₄, TiO₂ | |
| Na₈(AlSiO₄)₆(I, S)₂ | Zeolite A, NaI, | 5 * |
| | Na₂SO₄ | *Also 48 h |
| Na₈(AlSiO₄)₆(I, S)₂:Ti | Zeolite A, NaI, | 48 |
| | Na₂SO₄, TiO₂ | |
| K₂Na₆(AlSiO₄)₆(Cl, S)₂ | Zeolite A, KCl, | 5 |
| | Na₂SO₄ | |
| K₂Na₆(AlSiO₄)₆(Cl, | Zeolite A, KCl, | 48 |
| S)₂:Ti | Na₂SO₄, TiO₂ | |
| K₂Na₆(AlSiO₄)₆(Br, S)₂ | Zeolite A, KBr, | 5 |
| | Na₂SO₄ | |
| K₂Na₆(AlSiO₄)₆(Br, | Zeolite A, KBr, | 48 |
| S)₂:Ti | Na₂SO₄, TiO₂ | |
| K₂Na₆(AlSiO₄)₆(I, S)₂ | Zeolite A, KI, | 5 |
| | Na₂SO₄ | |
| K₂Na₆(AlSiO₄)₆(I, S)₂: | Zeolite A, KI, | 48 |
| Ti | Na₂SO₄, TiO₂ | |
| Rb₂Na₆(AlSiO₄)₆(Cl, S)₂ | Zeolite A, RbCl, | 5 |
| | Na₂SO₄ | |
| Rb₂Na₆(AlSiO₄)₆(Cl, | Zeolite A, RbCl, | 48 |
| S)₂:Ti | Na₂SO₄, TiO₂ | |
| Cs₂Na₆(AlSiO₄)₆(Br, S)₂ | Zeolite A, CsBr, | 5 |
| | Na₂SO₄ | |
| LiNa₇(AlSiO₄)₆(Cl, S)₂ | Zeolite A, LiCl, | 5 |
| | NaCl, Na₂SO₄ | |
| LiNa₆K(AlSiO₄)₆(Cl, | Zeolite A, LiCl, | 5 |
| S)₂ | KCl, Na₂SO₄ | |
| LiNa₆Rb(AlSiO₄)₆(Cl, | Zeolite A, LiCl, | 5 |
| S)₂ | RbCl, Na₂SO₄ | |
| LiNa₇(AlSiO₄)₆(Br, S)₂ | Zeolite A, LiBr, | 5* |
| | NaBr, Na₂SO₄ | *Also 72 h, |
| | | 48 h, 36 h, |
| | | 24 h, 12 h, |
| | | 2 h |
| LiNa₆K(AlSiO₄)₆(Br, | Zeolite A, LiBr, | 5 |
| S)₂ | KBr, Na₂SO₄ | |
| LiNa₆K(AlSiO₄)₆(Br, | Zeolite A, LiBr, | 48 |
| S)₂:Ti | KBr, Na₂SO₄, TiO₂ | |
| LiNa₆Cs(AlSiO₄)₆(Br, | Zeolite A, LiBr, | 5 |
| S)₂ | CsBr, Na₂SO₄ | |
| KNa₇(AlSiO₄)₆(Cl, S)₂ | Zeolite A, NaCl, | 5 |
| | KCl, Na₂SO₄ | |
| RbNa₇(AlSiO₄)₆(Cl, S)₂ | Zeolite A, NaCl, | 5 |
| | RbCl, Na₂SO₄ | |
| KNa₇(AlSiO₄)₆(Br, S)₂ | Zeolite A, NaBr, | 5 |
| | KBr, Na₂SO₄ | |
| KNa₇(AlSiO₄)₆(Br, S)₂: | Zeolite A, NaBr, | 48 |
| Ti | KBr, Na₂SO₄, TiO₂ | |
| CsNa₇(AlSiO₄)₆(Br, S)₂ | Zeolite A, NaBr, | 5 |
| | CsBr, Na₂SO₄ | |
| KNa₇(AlSiO₄)₆(I, S)₂ | Zeolite A, NaI, KI, | 5 |
| | Na₂SO₄ | |
| KNa₇(AlSiO₄)₆(I, S)₂:Ti | Zeolite A, NaI, KI, | 48 |
| | Na₂SO₄, TiO₂ | |
| Na₆KRb(AlSiO₄)₆(Cl, | Zeolite A, KCl, | 5 |
| S)₂ | RbCl, Na₂SO₄ | |
| Na₆KCs(AlSiO₄)₆(Br, | Zeolite A, KBr, | 5 |
| S)₂ | CsBr, Na₂SO₄ | |
| LiNa₆K (Al— | Zeolite A, LiCl, | 48 |
| SiO₄)₆(Cl, I, S)₂:Ti | KI, Na₂SO₄, TiO₂ | |
| LiNa₆K (Al— | Zeolite A, LiBr, | 48 |
| SiO₄)₆(Br, I, S)₂:Ti | KI, Na₂SO₄, TiO₂ | |
| LiNa₇ (Al- | Zeolite A, LiBr, | 48 |
| SiO₄)₆(Br, I, S)₂:Ti | NaI, Na₂SO₄, TiO₂ | |
| Na₈(AlSiO₄)₆(Cl, Br, | Zeolite A, NaCl, | 48 |
| S)₂:Ti | NaBr, Na₂SO₄, TiO₂ | |
| Na₈(AlSiO₄)₆(Br, S)₂:Ti | Zeolite A, NaBr, | 5 |
| | Na₂SO₄, TiO₂ | |
| Na₈(AlSiO₄)₆(Br, S)₂:W | Zeolite A, NaBr, | 5 |
| | Na₂SO₄, WS₂ | |
| Na₈(AlSiO₄)₆(Br, S)₂:Ba | Zeolite A, NaBr, | 5 |
| | Na₂SO₄, BaBr₂ | |
| Na₈(AlSiO₄)₆(I, S)₂:Ti | Zeolite A, NaI, | 5 |
| | Na₂SO₄, TiO₂ | |
| Na₈(AlSiO₄)₆(I, S)₂:W | Zeolite A, NaI, | 5 |
| | Na₂SO₄, WS₂ | |
| Na₈(AlSiO₄)₆(I, S)₂:Ba | Zeolite A, NaI, | 5 |
| | Na₂SO₄, BaBr₂ | |
| LiNa₆K(AlSiO₄)₆(Cl, | Zeolite A, LiCl, | 5 |
| S)₂:Ti | KCl, Na₂SO₄, TiO₂ | |
| LiNa₆K(AlSiO₄)₆(Cl, | Zeolite A, LiCl, | 5 |
| S)₂:W | KCl, Na₂SO₄, WS₂ | |
| LiNa₆K(AlSiO₄)₆(Cl, | Zeolite A, LiCl, | 5 |
| S)₂:Ba | KCl, Na₂SO₄, BaBr₂ | |
| LiNa₇₍AlSiO₄)₆(Br, | Zeolite A, LiBr, | 5 |
| S)₂:Ti | NaBr, Na₂SO₄, TiO₂ | |
| LiNa₇(AlSiO₄)₆(Br, | Zeolite A, LiBr, | 5 |
| S)₂:W | NaBr, Na₂SO₄, WS₂ | |
| LiNa₇(AlSiO₄)₆(Br, | Zeolite A, LiBr, | 5* |
| S)₂:Ba | NaBr, Na₂SO₄, BaBr₂ | *Also 72 h, |
| | | 48 h, 36 h, |
| | | 24 h, 12 h, |
| | | 2 h |
| LiNa₆K(AlSiO₄)₆(Br, | Zeolite A, LiBr, | 5 |
| S)₂:Ti | KBr, Na₂SO₄, TiO₂ | |
| LiNa₆K(AlSiO₄)₆(Br, | Zeolite A, LiBr, | 5 |
| S)₂:W | KBr, Na₂SO₄, WS₂ | |
| LiNa₆K(AlSiO₄)₆(Br, | Zeolite A, LiBr, | 5 |
| S)_{2:}Ba | KBr, Na₂SO₄, BaBr₂ | |
| Na₆KCs(AlSiO₄)₆(Br, | Zeolite A, KBr, | 5 |
| S)₂:Ti | CsBr, Na₂SO₄, TiO₂ | |
| Na₆KCs(AlSiO₄)₆(Br, | Zeolite A, KBr, | 5 |
| S)₂:W | CsBr, Na₂SO₄, WS₂ | |
| Na₆KCs(AlSiO₄)₆(Br, | Zeolite A, KBr, | 5 |
| S)_{2:}Ba | CsBr, Na₂SO₄, BaBr₂ | |
| LiNa₇(AlSiO₄)₆(Br, | Zeolite A, LiBr, | 5* |
| S)₂:Ba, W | NaBr, Na₂SO₄, BaBr₂, | *Also 72 h, |
| | WS₂ | 48 h, 36 h, |
| | | 24 h, 12 h, |
| | | 2 h |
| LiNa₆K(AlSiO₄)₆(Br, | Zeolite A, LiBr, | 5 h |
| S)₂:Ba, W | KBr, Na₂SO₄, BaBr₂, | |
| | WS₂ | |
| LiNa₇(AlSiO₄)₆(Br, | Zeolite A, LiBr, | 5* |
| S)₂:Sr | NaBr, Na₂SO₄, SrBr₂ | *Also 72 h, |
| | | 48 h, 36 h, |
| | | 24 h, 12 h, |
| | | 2 h |
| LiNa₇(AlSiO₄)₆(Br, | Zeolite A, LiBr, | 5* |
| S)₂:Sr, W | NaBr, Na₂SO₄, SrBr₂, | *Also 72 h, |
| | WS₂ | 48 h, 36 h, |
| | | 24 h, 12 h, |
| | | 2 h |
| LiNa₇(AlSiO₄)₆(Br, | Zeolite A, LiBr, | 5* |
| S)₂:Sr, Ba | NaBr, Na₂SO₄, SrBr₂, | *Also 72 h, |
| | BaBr₂ | 48 h, 36 h, |
| | | 24 h, 12 h, |
| | | 2 h |
| LiNa₇(AlSiO₄)₆(Br, | Zeolite A, LiBr, | 5 |
| S)₂:Sr, Cu | NaBr, Na₂SO₄, SrBr₂, | |
| | CuBr | |

When tested in a similar manner as above for example 2, it was noted that the above optically active materials could be used as detector material in image detectors for X-ray-based imaging techniques.

### Example 4—Testing of a Sample of the Material of LiNa₇(AlSiO₄)₆(Cl,S)₂

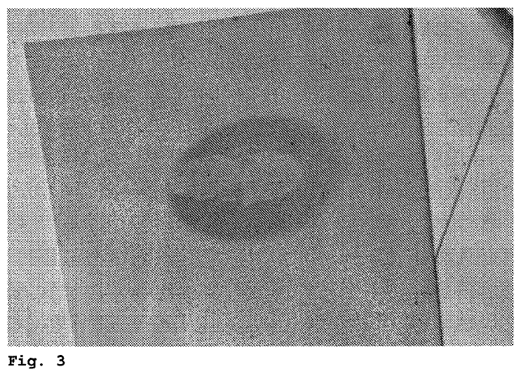

In this example a sample of LiNa₇(AlSiO₄)₆(Cl,S)₂ was subjected to X-ray imaging. For the X-ray imaging, the sample of the material was attached to the surface of a polymer film with tape casting technique using 300 μm wet thickness. An ant was put on top of an XRF machine's film that protects the equipment from material contamination. Right below the film is the source where the beam comes out. The prepared image detector or imaging plate was placed on top of the ant such that the ant was situated between the X-ray source and the imaging plate. Then the ant and the imaging plate were exposed to X-rays for 1 hour. The tenebrescence image of FIG. 3 that was produced from the exposure was photographed 28 times with a Nikon D5300. An image stacking program DeepSkyStacker and Photoshop Lightroom were used to bring out the details and contrast in the photo.

### Example 5—Testing of Samples of the Material of LiNa₇(AlSiO₄)₆(Br,S)₂:Sr and of LiNa₇(AlSiO₄)₆(Br,S)₂:Sr, Cu

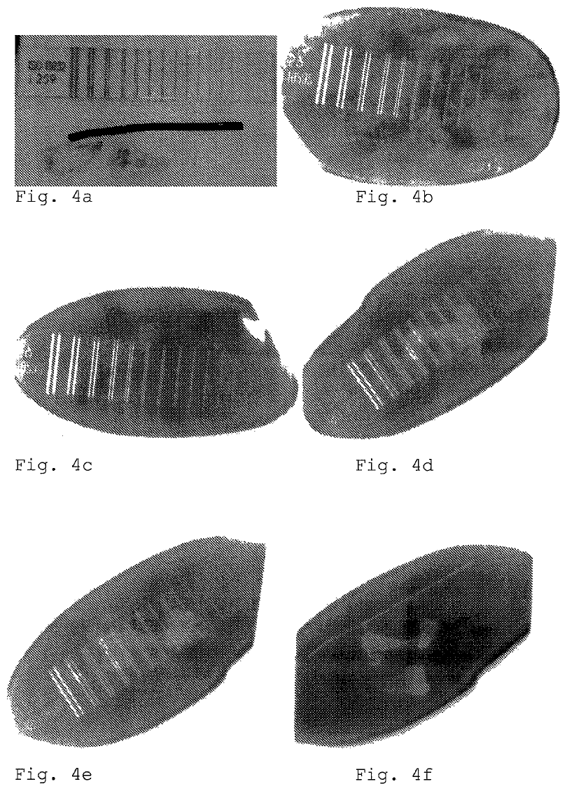

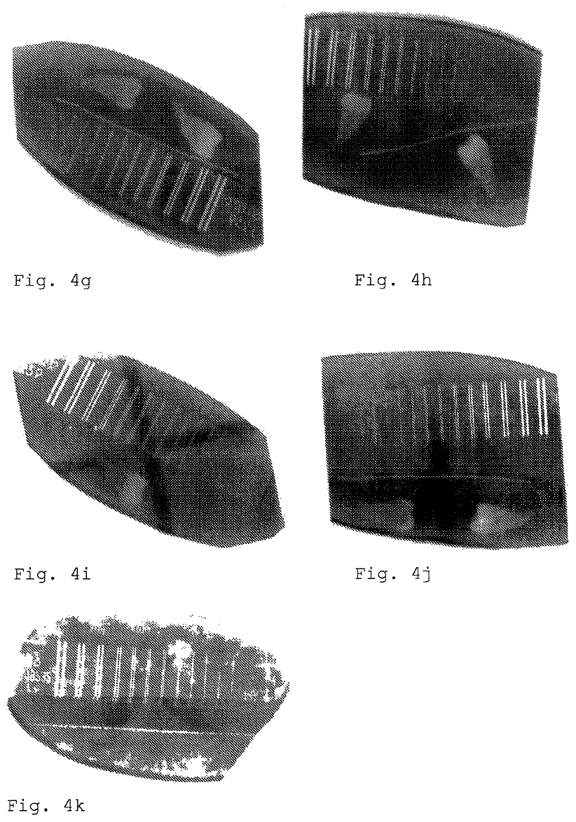

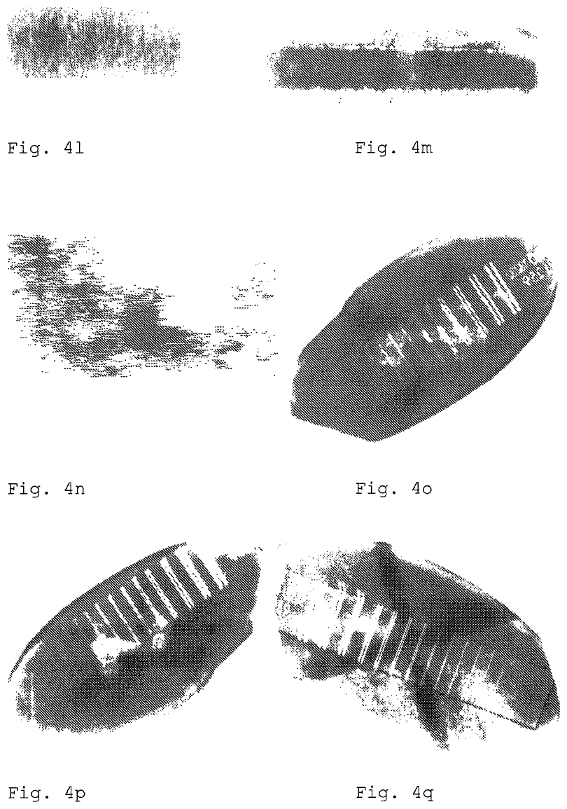

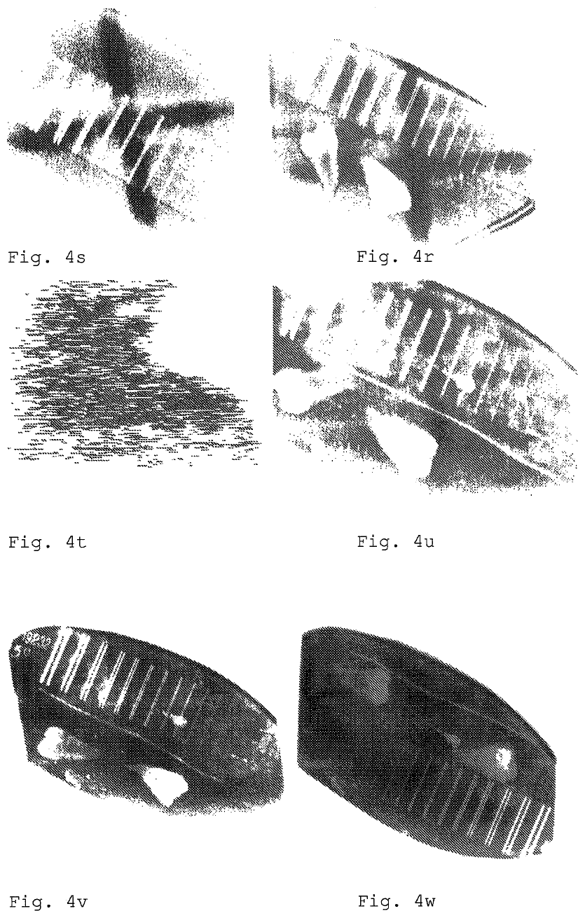

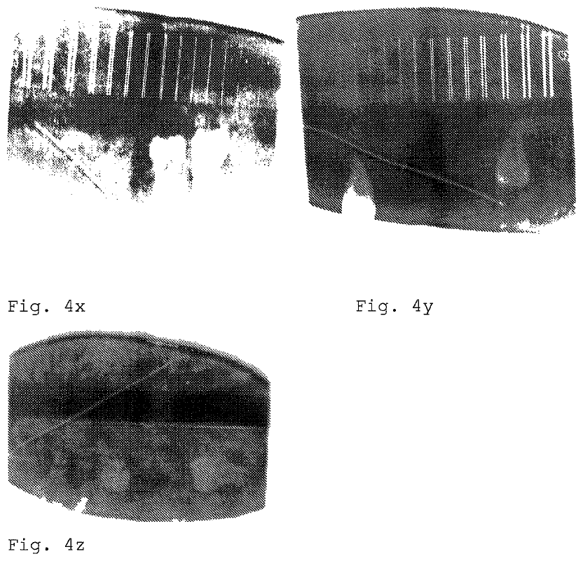

In this example samples of LiNa₇(AlSiO₄)₆(Br,S)₂:Sr and of LiNa₇(AlSiO₄)₆(Br,S)₂:Sr,Cu were subjected to X-ray imaging. For the X-ray imaging, the materials were attached to the surface of a polymer film using the same tape casting technique as in Example 1 using 300 μm wet thickness. The tapes were glued to a cardboard plate for support. Images were created using the X-ray beam of a Shimadzu MobileArt mobile X-ray system. A Duplex IQI standard, two human teeth and a plastic-coated metal wire were used as specimens to be imaged in the tests (see FIG. 4a). The image data was read with an unmodified intraoral X-ray image reader Durr Dental Vistascan using 635 nm stimulation. Some photographs of the specimens and X-ray images are shown in FIGS. 4b-4j. The testing parameters used are presented in the below table:

| | | | number of | | |
|---|---|---|---|---|---|
| Material | kV | mAs | exposures | mGy | FIGURE |
| LiNa₇(AlSiO₄)₆ | 100 | 28 | 10 | 179 | FIG. |
| (Br, S)₂:4% Sr | | | | | 4b |
| LiNa₇(AlSiO₄)₆ | 100 | 28 | 10 | 179 | FIG. |
| (Br, S)₂:6% Sr | | | | | 4c |
| LiNa₇(AlSiO₄)₆ | 80 | 25 | 10 | 102 | FIG. |
| (Br, S)₂:7% Sr | | | | | 4d |
| LiNa₇(AlSiO₄)₆ | 100 | 28 | 7 | 125 | FIG. |
| (Br, S)₂:7% Sr | | | | | 4e |
| LiNa₇(AlSiO₄)₆ | 100 | 28 | 10 | 179 | FIG. |
| (Br, S)₂:7% Sr | | | | | 4f |
| LiNa₇(AlSiO₄)₆ | 125 | 200 | 1 | 195 | FIG. |
| (Br, S)₂:6% Sr, | | | | | 4g |
| 1% Cu | | | | | |
| LiNa₇(AlSiO₄)₆ | 125 | 200 | 1 | 195 | FIG. |
| (Br, S)₂:6% Sr | | | | | 4h |
| LiNa₇(AlSiO₄)₆ | 100 | 28 | 10 | 179 | FIG. |
| (Br, S)₂:6% Sr, | | | | | 4i |
| 1% Cu | | | | | |
| LiNa₇(AlSiO₄)₆ | 100 | 28 | 7 | 125 | FIG. |
| (Br, S)₂:6% Sr | | | | | 43 |
| LiNa₇(AlSiO₄)₆ | 70 | 25 | 8 | 61 | FIG. |
| (Br, S)₂:3% Sr | | | | | 4k |
| LiNa₇(AlSiO₄)₆ | 70 | 20 | 4 | 24 | FIG. |
| (Br, S)₂:3% Sr | | | | | 4l |
| LiNa₇(AlSiO₄)₆ | 70 | 25 | 4 | 30 | FIG. |
| (Br, S)₂:3% Sr | | | | | 4m |
| LiNa₇(AlSiO₄)₆ | 70 | 25 | 8 | 61 | FIG. |
| (Br, S)₂:4% Sr | | | | | 4n |
| LiNa₇(AlSiO₄)₆ | 80 | 20 | 10 | 81 | FIG. |
| (Br, S)₂:7% Sr | | | | | 4o |
| LiNa₇(AlSiO₄)₆ | 70 | 25 | 8 | 61 | FIG. |
| (Br, S)₂:7% Sr | | | | | 4p |
| LiNa₇(AlSiO₄)₆ | 100 | 28 | 10 | 179 | FIG. |
| (Br, S)₂:4% Sr | | | | | 4q |
| LiNa₇(AlSiO₄)₆ | 70 | 25 | 8 | 61 | FIG. |
| (Br, S)₂:6% Sr, | | | | | 4r |
| 1% Cu | | | | | |
| LiNa₇(AlSiO₄)₆ | 80 | 20 | 10 | 81 | FIG. |
| (Br, S)₂:6% Sr, | | | | | 4s |
| 1% Cu | | | | | |
| LiNa₇(AlSiO₄)₆ | 100 | 10 | 10 | 63 | FIG. |
| (Br, S)₂:6% Sr, | | | | | 4t |
| 1% Cu | | | | | |
| LiNa₇(AlSiO₄)₆ | 80 | 25 | 10 | 102 | FIG. |
| (Br, S)₂:6% Sr, | | | | | 4u |
| 3% Cu | | | | | |
| LiNa₇(AlSiO₄)₆ | 100 | 28 | 10 | 179 | FIG. |
| (Br, S)₂:6% Sr, | | | | | 4v |
| 3% Cu | | | | | |
| LiNa₇(AlSiO₄)₆ | 125 | 200 | 1 | 195 | FIG. |
| (Br, S)₂:6% Sr, | | | | | 4w |
| 3% Cu | | | | | |
| LiNa₇(AlSiO₄)₆ | 100 | 10 | 10 | 63 | FIG. |
| (Br, S)₂:6% Sr | | | | | 4x |
| LiNa₇(AlSiO₄)₆ | 80 | 25 | 10 | 102 | FIG. |
| (Br, S)₂:6% Sr | | | | | 4y |
| LiNa₇(AlSiO₄)₆ | 100 | 28 | 10 | 179 | FIG. |
| (Br, S)₂:3% Sr | | | | | 4z |

### Example 6—Testing of Samples of the Material of LiNa₇(AlSiO₄)₆(Br,S)₂:Sr

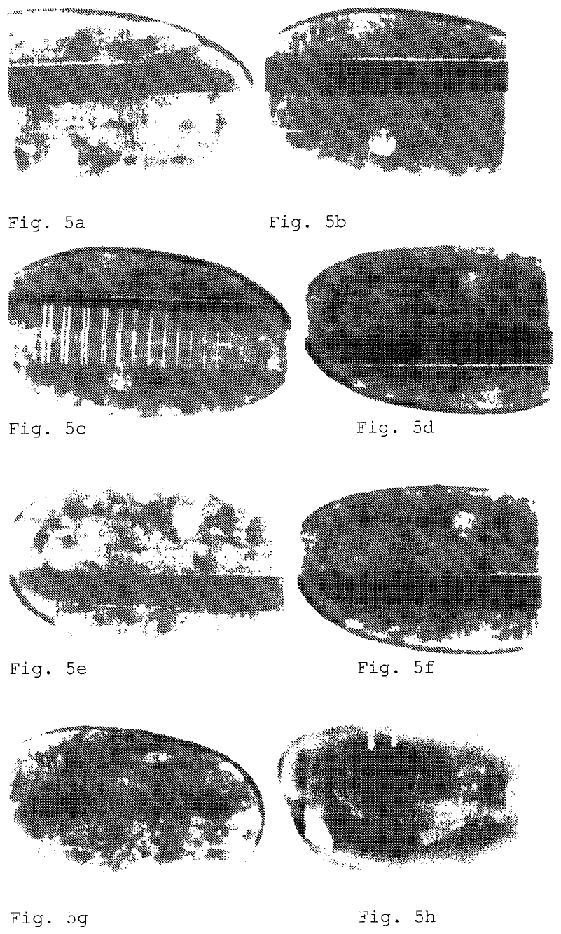

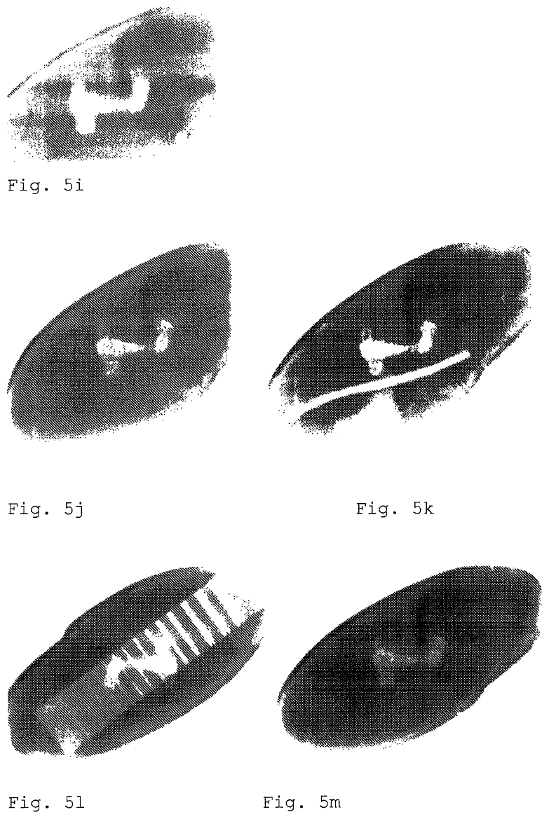

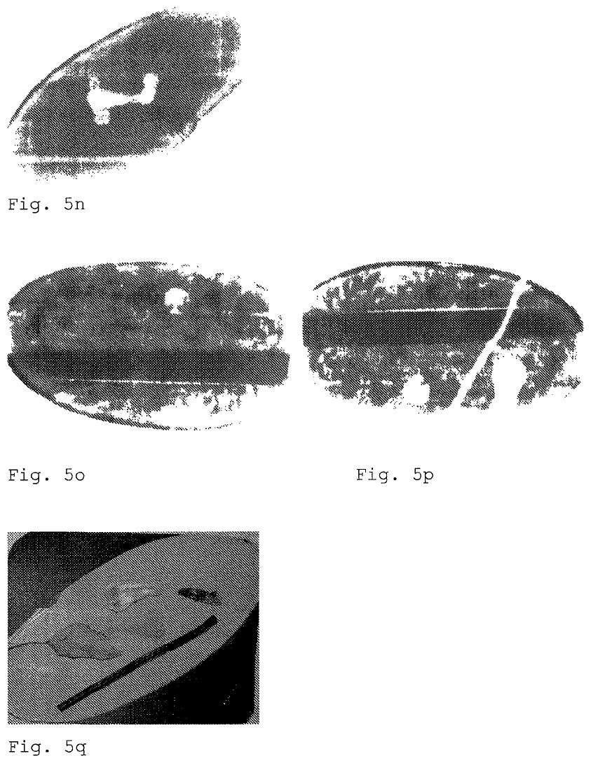

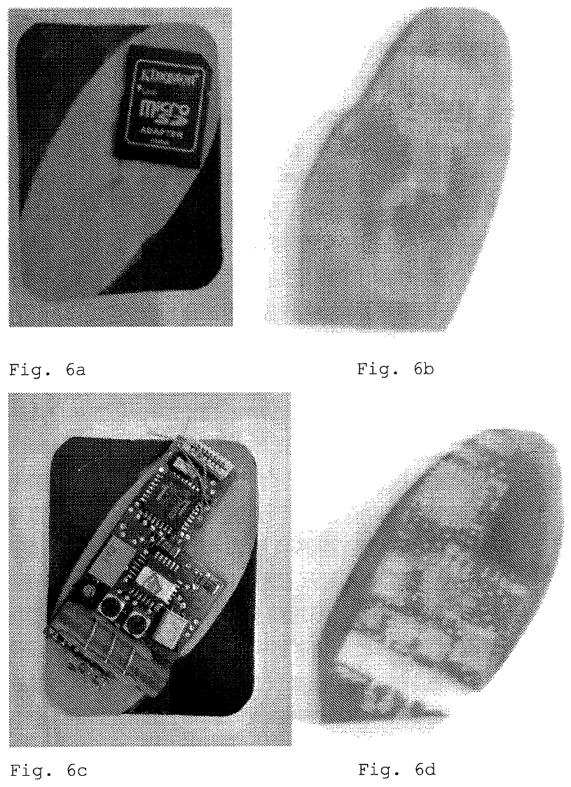

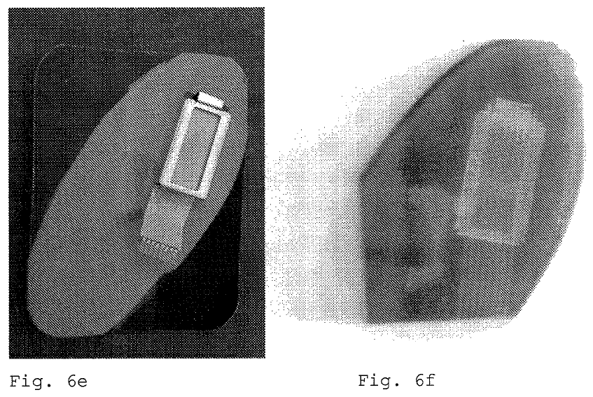

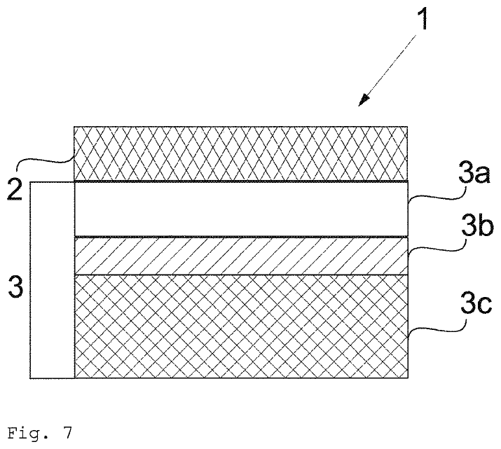

In this example samples of LiNa₇(AlSiO₄)₆(Br,S)₂:Sr were subjected to X-ray imaging. For the X-ray imaging, the materials were attacked to the surface of a polymer film using the same tape casting technique as in Example 1 using 300 μm wet thickness. The tapes were glued to a cardboard plate for support. Images were created using the X-ray beam of a Soredex Mamex dc mag mammography device. A Duplex IQI standard, a human tooth, a dead winged ant, a dead leaf and a plastic-coated metal wire were used as specimens to be imaged in the tests. The image data was read with an unmodified intraoral X-ray image reader Durr Dental Vistascan using 635 nm stimulation. Photographs of the specimens (FIG. 5q) and X-ray images (FIG. 5a-5p) are shown in FIG. 5. The testing parameters used are presented in the below table:

| | | | number | | |
|---|---|---|---|---|---|
| | | | of | | |
| Material | kV | mAs | exposures | mGy | FIGURE |
| LiNa₇(AlSiO₄)₆ | 25 | 100 | 2 | 39 | FIG. 5a |
| (Br, S)₂:3% Sr | | | | | |
| LiNa₇(AlSiO₄)₆ | 25 | 100 | 3 | 58 | FIG. 5b |
| (Br, S)₂:3% Sr | | | | | |
| LiNa₇(AlSiO₄)₆ | 25 | 100 | 4 | 77 | FIG. 5c |
| (Br, S)₂:3% Sr | | | | | (duplex IQI); |
| | | | | | FIG. 5d |
| | | | | | (ant&leaf |
| LiNa₇(AlSiO₄)₆ | 25 | 200 | 1 | 39 | FIG. 5e |
| (Br, S)₂:3% Sr | | | | | |
| LiNa₇(AlSiO₄)₆ | 25 | 320 | 1 | 62 | FIG. 5f |
| (Br, S)₂:3% Sr | | | | | |
| LiNa₇(AlSiO₄)₆ | 25 | 100 | 5 | 97 | FIG. 5g |
| (Br, S)₂:4% Sr | | | | | |
| LiNa₇(AlSiO₄)₆ | 25 | 100 | 5 | 97 | FIG. 5h |
| (Br, S)₂:6% Sr | | | | | |
| LiNa₇(AlSiO₄)₆ | 25 | 100 | 3 | 58 | FIG. 5i |
| (Br, S)₂:7% Sr | | | | | |
| LiNa₇(AlSiO₄)₆ | 25 | 100 | 4 | 77 | FIG. 5j |
| (Br, S)₂:7% Sr | | | | | (ant&leaf); |
| | | | | | FIG. 5k |
| | | | | | (tooth&plastic |
| | | | | | coated wire) |
| LiNa₇(AlSiO₄)₆ | 25 | 100 | 5 | 97 | FIG. 5l |
| (Br, S)₂:7% Sr | | | | | (Duplex IQI); |
| | | | | | FIG. 5m |
| | | | | | (ant&leaf) |
| LiNa₇(AlSiO₄)₆ | 25 | 320 | 1 | 62 | FIG. 5n |
| (Br, S)₂:7% Sr | | | | | |
| LiNa₇(AlSiO₄)₆ | 25 | 100 | 3 | 58 | FIG. 5o |
| (Br, S)₂:3% Sr | | | | | (ant&leaf); |
| | | | | | FIG. 5p |
| | | | | | (tooth@plastic- |
| | | | | | coated wire) |

Photographs of the specimens and X-ray images showed that the material can be used for mammography imaging or testing.

### Example 7—Testing of a Sample of the Material LiNa₇(AlSiO₄)₆(Br,S)₂:7% Sr

In this example a sample of LiNa₇(AlSiO₄)₆(Br,S)₂:7% Sr was subjected to X-ray imaging. For the X-ray imaging, the material was attached to the surface of a polymer film using the same tape casting technique as in Example 1 using 300 μm wet thickness. The tapes were glued to a cardboard plate for support. Images were created using the X-ray beam of a Shimadzu MobileArt mobile X-ray system. A microSD to SD memory card ad ter, a Contactor PY8205 pager machine's circuit board, and display unit were used as specimens to be imaged in the tests. The image data was read with an unmodified intraoral X-ray image reader Durr Dental Vistascan using 635 nm stimulation. Photographs of the specimens and X-ray images are shown in FIG. 6. The testing parameters used are presented in the below table:

| | | | number | | |
|---|---|---|---|---|---|
| | | | of | | |
| Material | kV | mAs | exposures | mGy | FIGURE |
| LiNa₇(AlSiO₄)₆ | 90 | 320 | 1 | 167 | FIGS. 6a and |
| (Br, S)₂:7% Sr | | | | | 6b |
| LiNa₇(AlSiO₄)₆ | 125 | 200 | 5 | 972 | FIGS. 6c and |
| (Br, S)₂:7% Sr | | | | | 6d |
| LiNa₇(AlSiO₄)₆ | 125 | 200 | 5 | 972 | FIGS. 6e and |
| (Br, S)₂:7% Sr | | | | | 6f |

It is obvious to a person skilled in the art that with the advancement of technology, the basic idea may be implemented in various ways. The embodiments are thus not limited to the examples described above; instead, they may vary within the scope of the claims.

The embodiments described hereinbefore may be used in any combination with each other. Several of the embodiments may be combined together to form a further embodiment. An image detector or a use, disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1. An image detector for a radiation-based imaging technique, wherein the image detector comprises a detector material on a substrate, wherein the detector material is an optically active material represented by the following formula (I)
(M)₈(M″M′″)₆O₂₄(X,X′)₂:M″″ formula (I)
wherein
M′ represents a monoatomic cation of an alkali metal selected from Group 1 of the IUPAC periodic table of the elements, or of an alkaline earth metal selected from Group 2 of the IUPAC periodic table of the elements, or any combination of such cations;
M″ represents a trivalent monoatomic cation of an element selected from Group 13 of the IUPAC periodic table of the elements, or of a transition element selected from any of Groups 3-12 of the IUPAC periodic table of the elements, or any combination of such cations;
M′″ represents a monoatomic cation of an element selected from Group 14 of the IUPAC periodic table of the elements, or of an element selected from any of Groups 13 and 15 of the IUPAC periodic table of the elements, or of Zn, or any combination of such cations;
X represents an anion of an element selected from Group 17 of the IUPAC periodic table of the elements, or any combination of such anions, or wherein X is absent;
X′ represents an anion of one or more elements selected from Group 16 of the IUPAC periodic table of the elements, or any combination of such anions, or wherein X′ is absent; and
M″″ represents a dopant cation of an element selected from rare earth metals of the IUPAC periodic table of the elements, or from transition metals of the IUPAC periodic table of the elements, or of Ba, Sr, TI, Pb, or Bi, or any combination of such cations, or wherein M″″ is absent;
with the proviso that at least one of X and X′ is present.

2. The image detector of claim 1, wherein M′ represents a monoatomic cation of an alkali metal selected from Group 1 of the IUPAC periodic table of the elements, or any combination of such cations, with the proviso that M′ does not represent the monoatomic cation of Na alone.

3. The image detector of claim 1, wherein M′ represents a combination of at least two monoatomic cations of different alkali metals selected from Group 1 of the IUPAC periodic table of the elements.

4. The image detector of claim 1, wherein M′ represents a combination of at least two monoatomic cations of different alkali metals selected from a group consisting of Li, Na, K, Rb, Cs, and Fr.

5. The image detector of claim 1, wherein M′ represents a monoatomic cation of a metal selected from a group consisting of Li, K, Rb, Cs, Fr, Be, Mg, Ca, Sr, Ba, Ra, or any combination of such cations.

6. The image detector of claim 1, wherein M′ represents a combination of at least two monoatomic cations of different metals, wherein at least one metal is selected from Group 1 of the IUPAC periodic table of the elements and at least one metal is selected from Group 2 of the IUPAC periodic table of the elements.

7. The image detector of claim 1, wherein M″ represents a trivalent monoatomic cation of a metal selected from a group consisting of Al and Ga, or a combination of such cations.

8. The image detector of claim 1, wherein M″ represents a trivalent monoatomic cation of B.

9. The image detector of claim 1, wherein M′″ represents a monoatomic cation of an element selected from a group consisting of Si and Ge, or a combination of such cations.

10. The image detector of claim 1, wherein M′″ represents a monoatomic cation of an element selected from a group consisting of Al, Ga, N, P, and As, or any combination of such cations.

11. The image detector of claim 1, wherein X represents an anion of an element selected from a group consisting of F, CI, Br, I, and At, or any combination of such anions.

12. The image detector of claim 1, wherein X′ represents a monoatomic or a polyatomic anion of one or more elements selected from a group consisting of O, S, Se, and Te, or any combination of such anions.

13. The image detector of claim 1, wherein M″″ represents a cation of an element selected from a group consisting of Yb, Er, Tb, and Eu, or any combination of such cations.

14. The image detector of claim 1, wherein M″″ represents a cation of an element selected from a group consisting of Ti, V, Cr, Mn, Fe, Co, Ni, Cu, Ag, W, and Zn, or any combination of such cations.

15. The image detector as defined in claim 1, wherein the radiation-based imaging technique is an X-ray-based imaging technique, a UV-radiation-based imaging technique, or a gamma-radiation-based imaging technique.

16. The image detector of claim 15, wherein the X-ray-based imaging technique is X-ray imaging, computed radiography (CR), digital radiography (DR), or computed tomography (CT).

17. A method of providing point-of-care analysis comprising providing an image detector including a detector material on a substrate, wherein the detector material is an optically active material represented by the following formula (I)
(M′)₈(M″M′″)₆O₂₄(X,X′)₂:M″″ formula (I)
wherein
M′ represents a monoatomic cation of an alkali metal selected from Group 1 of the IUPAC periodic table of the elements, or of an alkaline earth metal selected from Group 2 of the IUPAC periodic table of the elements, or any combination of such cations;
M″ represents a trivalent monoatomic cation of an element selected from Group 13 of the IUPAC periodic table of the elements, or of a transition element selected from any of Groups 3-12 of the IUPAC periodic table of the elements, or any combination of such cations;
M′″ represents a monoatomic cation of an element selected from Group 14 of the IUPAC periodic table of the elements, or of an element selected from any of Groups 13 and 15 of the IUPAC periodic table of the elements, or of Zn, or any combination of such cations;
X represents an anion of an element selected from Group 17 of the IUPAC periodic table of the elements, or any combination of such anions, or wherein X is absent;
X′ represents an anion of one or more elements selected from Group 16 of the IUPAC periodic table of the elements, or any combination of such anions, or wherein X′ is absent; and
M″″ represents a dopant cation of an element selected from rare earth metals of the IUPAC periodic table of the elements, or from transition metals of the IUPAC periodic table of the elements, or of Ba, Sr, TI, Pb, or Bi, or any combination of such cations, or wherein M″″ is absent; with the proviso that at least one of X and X′ is present; and further comprising:
performing an imaging technique on a patient.

18. A method of performing a radiation-based imaging technique comprising providing an image detector including detector material on a substrate, wherein the detector material is an optically active material represented by the following formula (I)
(M′)₈(M″M′″)₆O₂₄(X,X′)₂:M″″ formula (I)
wherein
M′ represents a monoatomic cation of an alkali metal selected from Group 1 of the IUPAC periodic table of the elements, or of an alkaline earth metal selected from Group 2 of the IUPAC periodic table of the elements, or any combination of such cations;
M″ represents a trivalent monoatomic cation of an element selected from Group 13 of the IUPAC periodic table of the elements, or of a transition element selected from any of Groups 3-12 of the IUPAC periodic table of the elements, or any combination of such cations;
M′″ represents a monoatomic cation of an element selected from Group 14 of the IUPAC periodic table of the elements, or of an element selected from any of Groups 13 and 15 of the IUPAC periodic table of the elements, or of Zn, or any combination of such cations;
X represents an anion of an element selected from Group 17 of the IUPAC periodic table of the elements, or any combination of such anions, or wherein X is absent;
X′ represents an anion of one or more elements selected from Group 16 of the IUPAC periodic table of the elements, or any combination of such anions, or wherein X′ is absent; and
M″″ represents a dopant cation of an element selected from rare earth metals of the IUPAC periodic table of the elements, or from transition metals of the IUPAC periodic table of the elements, or of Ba, Sr, TI, Pb, or Bi, or any combination of such cations, or wherein M″″ is absent; with the proviso that at least one of X and X′ is present; and further comprising:
performing the imaging technique.

19. The method of claim 17, wherein the radiation-based imaging technique is an X-ray-based imaging technique, a UV-radiation-based imaging technique, or a gamma-radiation-based imaging technique.

20. The method of claim 19, wherein the X-ray-based imaging technique is X-ray imaging, computed radiography (CR), digital radiography (DR), or computed tomography (CT).
